(19) United States
(12) Patent Application Publication
(10) Pub. No.: US 2007/0101513 A1
(43) Pub. Date: May 10, 2007

(54) Keratin fibers colorant with a nacreous luster

(76) Inventors: Manuela Javet, Marly (CH); Dominique Le Cruer, Farvagny (CH); Catherine Mueller, Marly (CH)

Correspondence Address: Striker Striker & Stenby, 103 East Neck Road, Huntington, NY, 11743, US

(21) Appl. No.: 10/578,751

(22) PCT Filed: Oct. 26, 2004
(86) PCT No.: PCT/EP04/12079

§ 371 Date: May 9, 2006

(30) Foreign Application Priority Data
Feb. 5, 2004 (DE) 10 2004 005 767.2

(51) Int. Cl. A61K 8/00

(52) U.S. Cl. 8/405

(57) ABSTRACT

The present application has for an object a dye carrier composition containing oxidative and/or nonoxidative dyes as well as (a) at least one fatty alcohol with 14 to 60 carbon atoms, (b) at least one alkanolamide, (c) at least one fatty ester and (d) at least one anionic surfactant, the weight ratio of fatty alcohol (a) to alkanolamide (b) being equal to 1:4 to 4:1. Another object is the use of the aforesaid combination of components (a) to (d) for creating a nacreous luster effect in dye carrier compositions and in colorants for keratin fibers.

The invention has for an object agents with a nacreous luster for coloring keratin fibers, particularly human hair, containing direct and/or oxidative dyes and a special combination of fatty alcohols, alkanolamides, fatty esters and anionic surfactants, as well as the use of the afore-said combination for creating a stable nacreous luster in the hair colorants.

Coloring preparations are usually in the form of aqueous—preferably thickened—solutions or emulsions and besides dyes contain, for example, fatty alcohols and/or other oil components, emulsifiers, surfactants and optionally alcohols. Oxidation dyes as a rule consist of two components, (i) the dye carrier composition containing the dyes and (ii) the oxidant preparation, which are mixed with one another just before use and then applied to the hair to be colored. If the coloring preparations are in the form of emulsions, they are as a rule stable creams, but to obtain a nacreous luster effect it is necessary to add to them special nacreous luster-imparting additives.

From DE-A 38 34 142 or EP-A 1 142 557 are known creamy hair colorants containing a multiplicity of raw materials including fatty alcohols and fatty alkanolamides as well as fatty esters, anionic surfactants and fatty alcohol alkoxylates. These colorants, however, do not have a nacreous luster. Moreover, fatty alcohol alkoxylates (PEG-based nonionic surfactants and emulsifiers) are under suspicion that they render hair more permeable thus trapping harmful substances. This possible property of fatty alcohol alkoxylates is worrisome particularly in colorants, because an increased penetration rate of dyes can lead to undesirable reactions in the body of the consumer.

The goal therefore was to develop an alkoxylate-free dye composition which without the addition of nacreous luster-imparting agents, by the selection of the raw materials alone, would exhibit stable nacreous luster-type character that would be retained even after mixing with the oxidant preparation. Moreover, the hair-care effect after rinsing out the dye composition was to be improved over prior-art formulations.

We have now found that this goal can be reached in outstanding manner by use of a combination of a fatty alcohol, an alkanolamide, a fatty ester and an anionic surfactant.

The present invention therefore has for an object a dye carrier composition containing oxidative and/or nonoxidative (“direct”) dyes, characterized in that it is free of fatty alcohol alkoxylates and contains a combination of
(a) at least one fatty alcohol with 14 to 60 carbon atoms,
(b) at least one alkanolamide,
(c) at least one fatty ester and
(d) at least one anionic surfactant,
   the weight ratio of fatty alcohol (a) to alkanolamide (b) being equal to 1:4 to 4:1.

Particularly preferred are dye carrier compositions for which the weight ratio of fatty alcohol (a) to alkanolamide (b) is equal to 1:1.7 to 2:1

Furthermore, to create an unusually beautiful nacreous luster-like character and an unusually high hair-care effect after the dye composition has been rinsed out, it is advantageous for the weight ratio of fatty ester (c) to the anionic surfactant (d) to be equal to 1:7 to 7:1, a (c):(d) weight ratio of 1:1.5 to 1.5:1 being particularly preferred.

According to the invention, suitable long-chain fatty alcohols with 14 to 60 carbon atoms are, for example, cetyl alcohol, stearyl alcohol, behenyl alcohol, myristyl alcohol, isooctyl alcohol or isotridecyl alcohol and mixtures of C₃₀-C₅₀ alcohols or C₄₀-C₆₀ alcohols. In the dye carrier composition of the invention, the fatty alcohols can be present alone or in combination with one another, the mixture of stearyl alcohol and behenyl alcohol with a weight ratio of 1:4 to 1:1 being particularly preferred.

Suitable alkanolamides according to the invention are, in particular, the N-acyl derivatives of monoethanolamine or diethanolamine, for example monoethanolamide and diethanolamide, or the ester amides such as coco fatty acid monoethanolamide.

Suitable fatty esters according to the invention are, for example, the monofatty acid or difatty acid esters of ethylene glycol, polyethylene glycol or glycerol, for example ethylene glycol distearate, glyceryl dioleate, glyceryl stearate, glyceryl distearate, glyceryl behenate and PEG-3 distearate, marketed, for example, under the trade name Tegin by Goldschmidt, Germany. Ethylene glycol distearate is particularly preferred.

Suitable anionic surfactants according to the invention are the salts and esters of carboxylic acids, alkyl ether sulfates and alkyl sulfates, fatty alcohol ether sulfates, sulfonic acid and the salts thereof (for example, sulfosuccinates or fatty acid isethionates), phosphate esters and the salts thereof and acylamino acids and the salts thereof. A detailed description of these anionic surfactants can be found in the publication “FIEDLER—Lexikon der Hilfsstoffe” [FIEDLER—Encyclopedia of Auxiliary Substances], vol. 1, fifth edition (2002), pages 97 to 102, to which we hereby specifically refer.

In the dye carrier composition of the invention, the fatty alcohol of component (a) and the alkanolamide of component (b) are used in a total amount from 6 to 20 weight percent each, a total amount from 7 to 15 weight percent each being preferred.

The fatty ester is used in a total amount from 0.1 to 15 weight percent and preferably from 7 to 12 weight percent, whereas the anionic surfactant is used in a total amount from 0.1 to 15 weight percent and preferably in an amount from 0.5 to 10 weight percent.

The dye carrier composition of the invention is preferably free of monomeric quaternary ammonium compounds and cationic emulsifier and surfactants.

Particularly preferred is an (a):(b) ratio from 1:1.7 to 2:1 which imparts an unusually beautiful nacreous luster character. A (c):(d) ratio from 1:7 to 7:1 and particularly from 1:1.5 to 6:1 is advantageous for creating the nacreous luster character and an unusual hair-care effect after the dye carrier composition has been rinsed out.

The dye carrier composition of the invention preferably contains oxidation dye precursors from which the coloration is created by the action of an oxidant, for example hydrogen peroxide and an adduct thereof, or in the presence of atmospheric oxygen.

Suitable oxidation dye precursors are, for example, the following developers, couplers and self-coupling compounds:
(i) Developers: 1,4-diaminobenzene (p-phenylenediamine), 1,4-diamino-2-methylbenzene (p-toluylenediamine), 1,4-diamino-2,6-dimethylbenzene, 1,4-diamino-3,5-diethylbenzene, 1,4-diamino-2,5-dimethylbenzene, 1,4-diamino-2,3-dimethylbenzene, 2-chloro-1,4-diaminobenzene, 1,4-diamino-2-(thiophen-2-yl)benzene, 1,4-diamino-2-(thiophen-3-yl)benzene, 1,4-diamino-2-(pyridin-3-yl)-benzene, 2,5-diaminobiphenyl, 1,4-diamino-2-methoxymethylbenzene, 1,4-diamino-2-aminomethylbenzene, 1,4-diamino-2-hydroxymethylbenzene, 1,4-diamino-2-(2-hydroxyethoxy)benzene, 2-[(2-acetylamino)ethoxy]-1,4-diaminobenzene, 4-phenylaminoaniline, 4-dimethylaminoaniline, 4-diethylaminoaniline, 4-dipropylaminoaniline, 4-[ethyl(2-hydroxyethyl)amino]aniline, 4-[di(2-hydroxyethyl)amino]aniline, 4-[di(2-hydroxyethyl)amino]-2-methylaniline, 4-[(2-methoxyethyl)amino]aniline, 4-[(3-hydroxypropyl)amino]aniline, 4-[(2,3-dihydroxypropyl)amino]aniline, 1,4-diamino-2-(2-hydroxyethyl)benzene, 1,4-diamino-2-(1-methylethyl)benzene, 1,3-bis[(4-aminophenyl)(2-hydroxyethyl)-amino]-2-propanol, 1,4-bis[(4-aminophenyl)amino]butane, 1,8-bis(2,5-diaminophenoxy)-3,6-dioxaoctane, 4-aminophenol, 4-amino-3-methylphenol, 4-amino-3-(hydroxymethyl)phenol, 4-amino-3-fluorophenol, 4-methylaminophenol, 4-amino-2-(aminomethyl)phenol, 4-amino-2-(hydroxymethyl)-phenol, 4-amino-2-fluorophenol, 4-amino-2-[(2-hydroxyethyl)amino]methylphenol, 4-amino-2-methylphenol, 4-amino-2-(methoxymethyl)phenol, 4-amino-2-(2-hydroxyethyl)phenol, 5-aminosalicylic acid, 2,5-diaminopyridine, 2,4,5,6-tetraaminopyrimidine, 2,5,6-triamino-4-(1H)-pyrimidone, 4,5-diamino-1-(2-hydroxyethyl)-1H-pyrazole, 4,5-diamino-1-(1-methylethyl)-1H-pyrazole, 4,5-diamino-1-[(4-methylphenyl)methyl]-1H-pyrazole, 1-[(4-chlorophenyl)methyl]-4,5-diamino-1H-pyrazole, 4,5-diamino-1-methyl-1H-pyrazole, 2-aminophenol, 2-amino-6-methylphenol and 2-amino-5-methylphenol, alone or in admixture with one another.
(ii) Couplers: N-(3-Dimethylaminophenyl)urea, 2,6-diaminopyridine, 2-amino-4-[(2-hydroxyethyl)amino]anisole, 2,4-diamino-1-fluoro-5-methylbenzene, 2,4-diamino-1-methoxy-5-methylbenzene, 2,4-diamino-1-ethoxy-5-methylbenzene, 2,4-diamino-1-(2-hydroxyethoxy)-5-methylbenzene, 2,4-di[(2-hydroxyethyl)amino]-1,5-dimethoxybenzene, 2,3-diamino-6-methoxypyridine, 3-amino-6-methoxy-2-(methylamino)pyridine, 2,6-diamino-3,5-dimethoxypyridine, 3,5-diamino-2,6-dimethoxy-pyridine, 1,3-diaminobenzene, 2,4-diamino-1-(2-hydroxyethoxy)benzene, 1,3-diamino-4-(2,3-dihy-droxypropoxy)benzene, 2,4-diamino-1,5-di(2-hydroxyethoxy)benzene, 1-(2-aminoethoxy)-2,4-di-aminobenzene, 2-amino-1-(2-hydroxyethoxy)-4-methylaminobenzene, 2,4-diaminophenoxyacetic acid, 3-[di(2-hydroxyethyl)amino]aniline, 4-amino-2-di[(2-hydroxyethyl)amino]-1-ethoxybenzene, 5-methyl-2-(1-methylethyl)phenol, 3-[(2-hydroxyethyl)amino]aniline, 3-[(2-aminoethyl)amino]aniline, 1,3-di(2,4-diaminophenoxy)propane, di(2,4-diaminophenoxy)methane, 1,3-diamino-2,4-dimethoxy-benzene, 2,6-bis(2-hydroxyethyl)aminotoluene, 4-hydroxyindole, 3-dimethylaminophenol, 3-dieth-ylaminophenol, 5-amino-2-methylphenol, 5-amino-4-fluoro-2-methylphenol, 5-amino-4-methoxy-2-methylphenol, 5-amino-4-ethoxy-2-methylphenol, 3-amino-2,4-dichlorophenol, 5-amino-2,4-dichlo-rophenol, 3-amino-2-methylphenol, 3-amino-2-chloro-6-methylphenol, 3-aminophenol, 2-[(3-hydroxyphenyl)amino]acetamide, 5-[(2-hydroxyethyl)amino]-4-methoxy-2-methylphenol, 5[(2-hydroxyethyl)amino]-2-methylphenol, 3[(2-hydroxyethyl)amino]phenol, 3-[(2-methoxyethyl)amino]phenol, 5-amino-2-ethylphenol, 5-amino-2-methoxyphenol, 2-(4-amino-2-hydroxyphenoxy)ethanol, 5-[(3-hydroxypropyl)amino]-2-methylphenol, 3-[(2,3-dihydroxypropyl)amino]-2-methylphenol, 3-[(2-hydroxyethyl)amino]-2-methylphenol, 2-amino-3-hydroxypyridine, 5-amino-4-chloro-2-methylphenol, 1-naphthol, 2-methyl-1-naphthol, 1,5-dihydroxynaphthalene, 1,7-dihydroxynaphthalene, 2,3-dihydroxynaphthalene, 2,7-dihydroxynaphthalene, 2-methyl-1-naphthol acetate, 1,3-dihydroxybenzene, 1-chloro-2,4-dihydroxybenzene, 2-chloro-1,3-dihydroxybenzene, 1,2-dichloro-3,5-dihydroxy-4-methylbenzene, 1,5-dichloro-2,4-dihydroxybenzene, 1,3-dihydroxy-2-methylbenzene, 3,4-methylenedioxyphenol, 3,4-methylenedioxyaniline, 5-[(2-hydroxyethyl)amino]-1,3-benzodioxol, 6-bromo-1-hydroxy-3,4-methylenedioxybenzene, 3,4-diaminobenzoic acid, 3,4-dihydro-6-hydroxy-1,4(2H)-benzoxazine, 6-amino-3,4-dihydro-1,4(2H)-benzoxazine, 3-methyl-1-phenyl-5-pyrazolone, 5,6-dihydroxyindole, 5,6-dihydroxyindoline, 5-hydroxyindole, 6-hydroxyindole, 7-hydroxyindole and 2,3-indolinedione, alone or in admixture with one another.
(iii) Self-coupling substances: 2-amino-5-methylphenol, 2-amino-6-methylphenol, 2-amino-5-ethoxyphenol or 2-propylamino-5-aminopyridine.

The total amount of the oxidation dye precursors contained in the dye carrier composition of the invention is about 0.01 to 12 weight percent and particularly about 0.2 to 6 weight percent.

To obtain certain color shades, common natural and/or synthetic direct dyes, for example vegetable dyes such as henna or indigo, triphenylmethane dyes, aromatic nitro dyes, azo dyes, quinone dyes and cationic or anionic dyes can also be contained in the colorant. Suitable synthetic dyes are, for example: 1,4-bis[(2-hydroxyethyl)amino]-2-nitrobenzene, 1-(2-hydroxyethyl)amino-2-nitro-4-[di(2-hydroxyethyl)amino]benzene (HC Blue No. 2), 1-amino-3-meth-yl-4-[(2-hydroxyethyl)amino]-6-nitrobenzene (HC Violet No. 1), 4-[ethyl-(2-hydroxyethyl)amino]-1-[(2-hydroxyethyl)amino]-2-nitrobenzene hydrochloride (HC Blue No. 12), 4-[di(2-hydroxyeth-yl)amino-]1-[(2-methoxyethyl)amino]-2-nitrobenzene (HC Blue No. 11), 1-[(2,3-dihydroxypropyl)-amino]-4-[methyl-(2-hydroxyethyl)amino]-2-nitrobenzene (HC Blue No. 10), 1-[(2,3-dihydroxy-propyl)amino]-4-[ethyl-(2-hydroxyethyl)amino]-2-nitrobenzene hydrochloride (HC Blue No. 9), 1-(3-hydroxypropylamino)-4-[di(2-hydroxyethyl)amino]-2-nitrobenzene (HC Violet No. 2), 1-methylami-no-4-[methyl-(2,3-dihydroxypropyl)amino]-2-nitrobenzene (HC Blue No. 6), 2-[(4-amino-2-nitro-phenyl)amino]-5-dimethylaminobenzoic acid (HC Blue No. 13), 1-amino-4-[(2-hydroxyethyl)amino]-2-nitrobenzene (HC Red No. 7), 2-amino-4,6-dinitrophenol, 4-amino-2-nitrodiphenylamine (HC Red No. 1), 1-amino-4-[di(2-hydroxyethyl)amino]-2-nitrobenzene hydrochloride (HC Red No. 13), 1-amino-5-chloro-4-[(2-hydroxyethyl)amino]-2-nitrobenzene, 4-amino-1-[(2-hydroxyethyl)ami-no]-2-nitrobenzene (HC Red No. 3), 4-amino-3-nitrophenol, 4-[(2-hydroxyethyl)amino]-3-nitro-phenol, 1-[(2-aminoethyl)amino]-4-(2-hydroxyethoxy)-2-nitrobenzene (HC Orange No. 2), 4-(2,3-dihydroxypropoxy)-1-[(2-hydroxyethyl)amino]-2-nitrobenzene (HC Orange No. 3), 1-amino-5-chlo-ro-4-[(2,3-dihydroxypropyl)amino]-2-nitrobenzene (HC Red No. 10), 5-chloro-1,4-[di(2,3-dihy-droxypropyl)amino]-2-nitrobenzene (HC Red No. 11), 2-[(2-hydroxyethyl)amino]-4,6-dinitrophe-nol, 4-ethylamino-3-nitrobenzoic acid, 2-[(4-amino-2-nitrophenyl)amino]benzoic acid, 2-chloro-6-ethylamino-4-nitrophenol, 2-amino-6-chloro-4-nitrophenol, 4-[(3-hydroxypropyl)amino]-3-nitrophe-nol, 2,5-diamino-6-nitropyridine, 1,2,3,4-tetrahydro-6-nitroquinoxaline, 7-amino-3,4-dihydro-6-nitro-2H-1,4-benzoxazine, (HC Red. No. 14), 1-amino-2-[(2-hydroxyethyl)amino]-5-nitrobenzene (HC Yellow No. 5), 1-(2-hydroxyethoxy)-2-[(2-hydroxy-ethyl)amino]-5-nitrobenzene (HC Yellow No. 4), 1-[(2-hydroxyethyl)amino]-2-nitrobenzene (HC Yellow No. 2), 2-[(2-hydroxyethyl)amino]-1-meth-oxy-5-nitrobenzene, 2-amino-3-nitrophenol, 1-(2-hydroxyethoxy)-3-methylamino-4-nitrobenzene, 2,3-(dihydroxypropoxy)-3-methylamino-4-nitrobenzene, 2-[(2-hydroxyethyl)amino]-5-nitrophenol (HC Yellow No. 11), 3-[(2-aminoethyl)amino]-1-methoxy-4-nitrobenzene hydrochloride (HC Yellow No. 9), 1-[(2-ureidoethyl)amino]-4-nitrobenzene, 4-[(2,3-dihydroxypropyl)amino]-3-nitro-1-trifluoromethylbenzene (HC Yellow No. 6), 1-chloro-2,4-bis[(2-hydroxyethyl)amino]-5-nitrobenzene (HC Yellow No. 10), 4-[(2-hydroxyethyl)amino]-3-nitro-1-methylbenzene, 1-chloro-4-[(2-hydroxyethyl)-amino]-3-nitrobenzene (HC Yellow No. 12), 4-[(2-hydroxyethyl)amino]-3-nitro-1-trifluoromethylbenzene (HC Yellow No. 13), 4-[(2-hydroxyethyl)amino]-3-nitrobenzonitrile (HC Yellow No. 14), 4-[(2-hydroxyethyl)amino]-3-nitrobenzamide (HC Yellow No. 15), 1,4-di[(2,3-dihydroxypropyl)amino]-9,10-anthraquinone, 1-[(2-hydroxyethyl)amino]-4-methylamino-9,10-anthraquinone (CI 61505, Disperse Blue No. 3), 2-[(2-aminoethyl)amino]-9,10-anthraquinone (HC Orange No. 5), 1-hydroxy-4-[(4-methyl-2-sulfophenyl)amino]-9,10-anthraquinone, 1-[(3-aminopropyl)amino]-4-methylamino-9,10-anthraquinone (HC Blue No. 8), 1-[(3-aminopropyl)amino]-9,10-anthraquinone (HC Red No. 8), 1,4-diamino-2-methoxy-9,10-anthraquinone (CI 62015, Disperse Red No. 11, Solvent Violet No. 26), 1,4-dihydroxy-5,8-bis[(2-hydroxyethyl)amino]-9,10-anthraquinone (CI 62500, Disperse Blue No. 7, Solvent Blue No. 69), 9-(dimethylamino)benzo[a]-phenoxazin-7-ium chloride (CI 51175; Basic Blue No. 6), di[4-(diethylamino)phenyl][4-(ethylamino)naphthyl]carbenium chloride (CI 42595; Basic Blue No. 7), 3,7-di(dimethylamino)phenothiazin-5-ium chloride (CI 52015; Basic Blue No. 9), di[4-(dimethylamino)phenyl][4-(phenylamino)naphthyl]carbenium chloride (CI 44045; Basic Blue No. 26), 2-{[4-(ethyl(2-hydroxyethyl)amino)phenyl]azo}-6-methoxy-3-methylbenzothiazolium methylsulfate (CI 11154; Basic Blue No. 41), 8-amino-2-bromo-5-hydroxy-4-imino-6-{[3-(trimethylammonio)phenyl]amino}-1(4H-naphthalenone chloride (CI 56059; Basic Blue No. 99), bis[4-(dimethylamino)phenyl][4-(methylamino)phenyl]carbenium chloride (CI 42535; Basic Violet No. 1), tris[4-(dimethylamino)phenyl]carbenium chloride (CI 42555; Basic Violet No. 3), 2-[3,6-(diethylamino)dibenzopyranium-9-yl]benzoyl chloride (CI 45170; Basic Violet No. 10), di(4-aminophenyl)(4-amino-3-methylphenyl)carbenium chloride (CI 42510, Basic Violet No. 14), 1,3-bis[(2,4-diamino-5-methylphenyl)azo]-3-methylbenzene (CI 21010; Basic Brown No. 4), 1-[(4-aminophenyl)azo]-7-(trimethylammonio)-2-naphthol chloride (CI 12250; Basic Brown No. 16), 1-[(4-amino-2-nitrophenyl)azo]-7-(trimethylammonio-2-naphthol chloride (Basic Brown No. 17), 1-[(4-amino-3-nitrophenyl)azo]-7-(trimethylammonio)-2-naphthol chloride (CI 12251; Basic Brown No. 17 [sic]), 3,7-diamino-2,8-dimethyl-5-phenylphenazinium chloride (CI 50240; Basic Red No. 2), 1,4-dimethyl-5-{[(4-(dimethylamino)phenyl]azo}-1,2,4-triazolium chloride (CI 11055; Basic Red No. 22), 2-hydroxy-1-[(2-methoxyphenyl)azo]-7-(trimethylammonio)naphthalene chloride (CI 12245; Basic Red No. 76), 2-{2-[(2,4-dimethoxyphenyl)amino]ethenyl}-1,3,3-trimethyl-3H-indol-1-ium chloride (CI 48055; Basic Yellow No. 11), 3-methyl-1-phenyl-4-{[3-(trimethylammonio)phenyl]azo}pyrazol-5-one chloride (CI 12719; Basic Yellow No. 57), bis[4-(diethylamino)phenyl]phenylcarbenium hydrogen sulfate (1:1) (CI 42040; Basic Green No. 1), 1-[di(2-hydroxyethyl)amino]-3-methyl-4-[(4-nitrophenyl)azo]benzene (CI 11210; Disperse Red No. 17), 4-[(4-aminophenyl)azo]-1-[di(2-hydroxyethyl)amino]-3-methylbenzene (HC Yellow No. 7), 2,6-diamino-3-[(pyridin-3-yl)azo]pyridine, 6-hydroxy-5-[(4-sulfophenyl)azo]-2-naphthalenesulfonic acid disodium salt (CI 15985; Food Yellow No. 3; FD&C Yellow No. 6), 2,4-dinitro-1-naphthol-7-sulfonic acid disodium salt (CI 10316; Acid Yellow No. 1; Food Yellow No. 1), 2-(indan-1,3-dion-2-yl)-quinoline-x,x-sulfonic acid (mixture of mono- and disulfonic acid) (CI 47005; D&C Yellow No. 10; Food Yellow No. 13; Acid Yellow No. 3), 5-hydroxy-1-(4-sulfophenyl)-4-[(4-sulfophenyl)azo]-pyrazole-3-carboxylic acid trisodium salt (CI 19140; Food Yellow No. 4; Acid Yellow No. 23), 9-(2-carboxyphenyl)-6-hydroxy-3H-xanthen-3-one (CI 45350; Acid Yellow No. 73; D&C Yellow No. 8), 5-[(2,4-dinitrophenyl)amino]-2-phenylaminobenzenesulfonic acid sodium salt (CI 10385; Acid Orange No. 3), 4-[(2,4-dihydroxyphenyl)azo]benzenesulfonic acid monosodium salt (CI 14270; Acid Orange No. 6), 4-[(2-hydroxynaphth-1-yl)azo]benzenesulfonic acid sodium salt (CI 15510; Acid Orange No. 7), 4-[(2,4-dihydroxy-3-[(2,4-dimethylphenyl)azo]phenyl)azo]benzenesulfonic acid sodium salt (CI 20170; Acid Orange No. 24), 4-hydroxy-3-[(4-sulfonaphth-1-yl)azo]-1-naphthalenesulfonic acid disodium salt (CI 14720; Acid Red No. 14), 6-hydroxy-5-[(4-sulfonaphth-1-yl)azo]-2,4-naphthalenedisulfonic acid trisodium salt (CI 16255; Ponceau 4R; Acid Red No. 18), 3-hydroxy-4-[(4-sulfonaphth-1-yl)azo]-2,7-naphthalenedisulfonic acid trisodium salt (CI 16185; Acid Red No. 27), 8-amino-1-hydroxy-2-(phenylazo)-3,6-naphthalenedisulfonic acid disodium salt (CI 17200; Acid Red No. 33), 5-(acetylamino)-4-hydroxy-3-[(2-methylphenyl)azo]-2,7-naphthalenedisulfonic acid disodium salt (CI 18065; Acid Red No. 35), 2-(3-hydroxy-2,4,5,7-tetraiododibenzopyran-6-on-9-yl)benzoic acid disodium salt (CI 45430; Acid Red No. 51), N-[6-(diethylamino)-9-(2,4-disulfophenyl)-3H-xanthen-3-ylidene]-N-ethylethanammonium hydroxide, inner salt, sodium salt (CI 45100; Acid Red No. 52), 8-{[4-(phenylazo)phenyl]azo}-7-naphthol-1,3-disulfonic acid disodium salt (CI 27290; Acid Red No. 73), 2′,4′,5′,7′-tetrabromo-3′,6′-dihydroxyspiro{isobenzofuran-1(3H),9′-[9H]-xanthen}-3-one disodium salt (CI 45380; Acid Red No. 87), 2′,4′,5′,7′-tetrabromo-4,5,6,7-tetrachloro-3′,6′-dihydroxyspiro{isobenzofuran-1(3H), 9′-[9H]-xanthen}-3-one disodium salt (CI 45410; Acid Red No. 92), 3′,6′-dihydroxy-4′,5′-diiodospiro[isobenzofuran-1(3H),9′(9H)xanthen]-3-one disodium salt (CI 45425; Acid Red No. 95), (2-sulfophenyl)di[4-(ethyl-((4-sulfophenyl)methyl)amino)phenyl]carbenium disodium salt betaine (CI 42090; Acid Blue No. 9; FD&C Blue No. 1), 1,4-bis[(2-sulfo-4-methylphenyl)amino]-9,10-anthraquinone disodium salt (CI 61570; Acid Green No. 25), bis[4-(dimethylamino)phenyl]-3,7-disulfo-2-hydroxynaphth-1-yl)car-benium inner salt, monosodium salt (CI 44090; Food Green No. 4; Acid Green No. 50), bis[4-(di-ethylamino)phenyl][2,4-disulfophenyl)carbenium inner salt, sodium salt (2:1) (CI 42045; Food Blue No. 3; Acid Blue No. 1), bis[4-(diethylamino)phenyl](5-hydroxy-2,4-disulfophenyl)carbenium inner salt, calcium salt (2:1) (CI 42051; Acid Blue No. 3), 1-amino-4-(cyclohexylamino)-9,10-anthraquinone-2-sulfonic acid sodium salt (CI 62045; Acid Blue No. 62), 2-(1,3-dihydro-3-keto-5-sulfo-2H-indol-2-ylidene)-2,3-dihydro-3-keto-1H-indole-5-sulfonic acid di-sodium salt (CI 73015; Acid Blue No. 74), 9-(2-carboxyphenyl)-3-[(2-methylphenyl)amino]-6-[(2-methyl-4-sulfophenyl)amino]-xanthylium inner salt, monosodium salt (CI 45190; Acid Violet No. 9), 1-hydroxy-4-[(4-methyl-2-sulfophenyl)amino]-9,10-anthraquinone sodium salt (CI 60730; D&C Violet No. 2; Acid Violet No. 43), bis{3-nitro-4-[(4-phenylamino)-3-sulfophenylamino]phenyl} sulfone (CI 10410; Acid Brown No. 13), 5-amino-4-hydroxy-6-[(4-nitrophenyl)azo]-3-(phenylazo)-2,7-naphthalenedisulfonic acid disodium salt (CI 20470; Acid Black No. 1), 3-hydroxy-4-[(2-hydroxynaphth-1-yl)azo]-7-nitro-1-naphthalenesulfonic acid chromium complex (3:2) (CI 15711; Acid Black No. 52), 3-[(2,4-dimethyl-5-sulfophenyl)azo]-4-hydroxy-1-naphthalenesulfonic acid disodium salt (CI 14700; Food Red No. 1; FD&C Red No. 4), 4-(acetylamino)-5-hydroxy-6-[(7-sulfo-4-[(4-sulfophenyl)azo]naphth-1-yl)azo]-1,7-naphthalenedisulfonic acid tetrasodium salt (CI 28440; Food Black No. 1) and 3-hydroxy-4-(3-methyl-5-keto-1-phenyl-4,5-dihydro-1H-pyrazol-4-ylazo)naphthalene-1-sulfonic acid sodium salt, chromium complex (Acid Red No. 195), alone or in combination with one another

The total amount of direct dyes in the dye carrier composition of the invention is about 0.01 to 7 weight percent and preferably about 0.2 to 4 weight percent.

Other common dyes known to be used for hair coloring and which can be contained in the colorant of the invention are described in, among other publications, E. Sagarin, “Cosmetics, Science and Technology”, lnterscience Publishers Inc., New York (1957), pages 503 ff, in H. Janistyn, “Handbuch der Kosmetika und Riechstoffe” [Handbook of Cosmetics and Fragrances], vol. 3 (1973), pages 388 ff and in K. Schrader “Grundlagen und Rezepturen der Kosmetika” [Fundamentals and Formulations of Cosmetics] 2nd edition, (1989), pages 782 to 815, to which we hereby specifically refer.

Although oxidative colorants are preferred, nonoxidative colorants based on the afore-said direct dyes can, of course, also be prepared with the dye carrier composition of the invention.

Furthermore, the dye carrier composition of the invention can contain antioxidants, for example ascorbic acid, thioglycolic acid or sodium sulfite, as well as complexing agents for heavy metals, for example an ethylenediaminetetraacete or nitriloacetic acid, in an amount of up to about 0.5 weight percent. Perfume oils can be contained in the dye carrier composition of the invention in an amount of up to about 1 weight percent. Naturally, the afore-described dye carrier composition can optionally also contain other additives commonly used in hair colorants, for example thickeners such as, for example, the homopolymers of acrylic acid, vegetable gums, derivatives of cellulose and starch, algal polysaccharides, amphiphilic associative thickeners, moreover preservatives, antioxidants, for example sodium sulfite, thioglycolic acid or ascorbic acid; complexing agents; solvents such as water, the lower aliphatic alcohols, for example aliphatic alcohols with 1 to 4 carbon atoms such as ethanol, propanol and isopropanol, or glycols such as glycerol and 1,2-propylene glycol; wetting agents or emulsifiers not cited in the main claims and belonging to the classes of anionic, amphoteric or nonionic surface-active agents; moreover softeners, vaseline, silicone oils, paraffin oil, polysorbates and fatty acids as well as hair-care agents such as cationic polymers or resins, lanolin derivatives, cholesterol, vitamins, pantothenic acid and betaine. The said constituents are used in amounts normally employed for such purposes, for example the wetting agents and emulsifiers at a concentration of 0.1 to 30 weight percent and the hair-care agents at a concentration of 0.1 to 5.0 weight percent.

For nonoxidative colorants based on direct dyes, the pH of the dye carrier composition of the invention is in the range from about 5 to 10 and preferably from 6 to 9, whereas for oxidative colorants based on oxidation dye precursors the pH is in the range from about 6 to 12 and preferably from 9 to 11, the pH of the ready-to-use oxidation hair colorant (namely the mixture of the dye carrier composition of the invention and the oxidant) being about 5.5 to 10, preferably 6 to 9.

Depending on the composition and the pH desired, the pH is adjusted preferably with ammonia, an amino acid or an organic amine, for example a glucamine, aminomethylpropanol, monoethanolamine or triethanolamine, an inorganic base, for example sodium hydroxide, potassium hydroxide, sodium carbonate or calcium hydroxide, or with an organic or inorganic acid, for example lactic acid, citric acid, acetic acid or phosphoric acid.

The dye carrier composition of the invention is preferably packaged in the form of an aqueous or aqueous-alcoholic preparation, for example as a thickened solution, emulsion, cream or gel.

For oxidative coloring, the afore-described dye carrier composition is mixed with an oxidant just before use, and an amount of the ready-to-use preparation sufficient for the coloring, as a rule about 60 to 200 grams, is applied to the fibers.

If the dye carrier composition of the invention contains no oxidation dye presursors or contains oxidation dye precursors that are readily oxidized by atmospheric oxygen, it can be applied to the keratin fibers directly without previous mixing with an oxidant.

Suitable oxidants for developing the coloration are mainly hydrogen peroxide or the addition compounds thereof to urea, melamine or sodium borate in the form of a 1 to 12 percent, preferably 1.5 to 6 percent aqueous solution. The mixing ratio of dye carrier composition to oxidant depends on the concentration of the oxidant and as a rule is about 5:1 to 1:3 and preferably 1:1, the oxidant being contained in the ready-to-use preparation preferably in an amount from about 0.5 to 8 wt. % and particularly from 1 to 4 weight percent.

The ready-to-use colorant is allowed to act on the keratin fibers (for example on human hair) at 15 to 50° C. for about 10 to 45 minutes and preferably for about 15 to 30 minutes after which the fibers are rinsed with water. Optionally, following this rinsing the fibers are washed with a shampoo and possibly post-rinsed with a weak organic acid, for example tartaric acid. The keratin fibers are then dried.

The dye carrier composition of the invention has a uniform consistency and produces a highly cosmetic nacreous luster effect. A colorant prepared with the dye carrier composition of the invention meets the requirements in terms of adhesion, application performance and viscosity adjustment in outstanding manner and through the nacreous character provides a highly cosmetic appearance. In addition, compared to known colorants a clearly improved hair-care result is attained after the rinsing

Another object of the present application is the use of a combination of
(a) at least one fatty alcohol with 14 to 60 carbon atoms,
(b) at least one alkanolamide,
(c) at least one fatty ester and
(d) at least one anionic surfactant
   the weight ratio of fatty alcohol (a) to alkanolamide (b) being equal to 1:4 to 4:1, for producing a nacreous luster effect in dye carrier compositions and in colorants for keratin fibers, particularly human hair.

Particularly preferred is the use of a combination of
(a) 6 to 20 weight percent, particularly 7 to 15 weight percent, of at least one fatty alcohol with 14 to 60 carbon atoms,
(b) 6 to 20 weight percent, particularly 7 to 15 weight percent, of at least one alkanolamide,
(c) 0.1 to 15 weight percent, particularly 7 to 12 weight percent, of at least one fatty ester and
(d) 0.1 to 15 weight percent and particularly 0.5 to 10 weight percent of at least one anionic surfactant,
   the weight ratio of fatty alcohol (a) to alkanolamide (b) being equal to 4:1 to 2:1 and particularly to 1:1.7 to 2:1.

The following examples will explain the subject matter of the invention in greater detail without limiting it to the examples.

### EXAMPLES

EXAMPLE 1
Oxidation Hair Colorant, Creamy

| | |
|---|---|
| 3.50 g | of stearyl alcohol |
| 7.50 g | of behenyl alcohol |
| 0.50 g | of mixture of C₃₀-C₅₀ alcohols (Performacol 550, supplied |
| | by Newphase) |
| 8.00 g | of coco fatty acid monoethanolamide (Cocamide MEA) |
| 9.50 g | of ethylene glycol distearate |
| 4.50 g | of sodium lauryl alcohol diethylene glycol ether sulfate, |
| | 28% aqueous solution |
| 2.00 g | of coco fatty acid isethionate |
| 0.60 g | of 2,5-diaminotoluene sulfate |
| 0.30 g | of resorcinol |
| 0.03 g | of m-aminophenol |
| 0.03 g | of 2-methylresorcinol |
| 9.00 g | of ammonia, 25% aqueous solution |
| 0.30 g | of disodium ethylenediaminetetraacetate |
| 0.40 g | of ascorbic acid |
| 0.30 g | of cationic cellulose derivative (Polyquaternium-10) |
| to 100.00 g | water |

Just before use, 50 g of the foregoing dye carrier composition with a nacreous luster was mixed with 50 g of a 6% aqueous hydrogen peroxide solution. This gave a homogeneous, cosmetically attractive dye preparation with a nacreous luster. The resulting mixture was then applied to blond hair. After an exposure time of 30 minutes at 40° C., the hair was rinsed with water and dried.

The hair thus received a medium-blond coloration.

EXAMPLE 2
Creamy Oxidation Hair Colorant

| Component (A): | Creamy Dye Carrier Composition |
|---|---|
| 11.000 g | of stearyl alcohol |
| 1.000 g | of behenyl alcohol |
| 8.500 g | of coco fatty acid monoethanolamide (Cocamide |
| | MEA) |
| 8.500 g | of ethylene glycol distearate |
| 1.800 g | of sodium myristyl sulfate (70% aqueous solution) |
| 2.000 g | of coco fatty acid isethionate |
| 3.000 g | of lauryl glucoside, 50% aqueous solution |
| | (Plantacare 1200 UP, supplied by Cognis) |
| 0.100 g | of 2,5-diaminotoluene sulfate |
| 0.040 g | of resorcinol |
| 0.006 g | of m-aminophenol |
| 0.014 g | of amino-4-hydroxyethylaminoanisole sulfate |
| 0.500 g | of disodium ethylenediaminetetraacetate |
| 16.000 g | of ammonia, 25% aqueous solution |
| 1.000 g | of ethanol |
| 3.000 g | of dimethyldiallylammonium chloride/acrylamide |
| | copolymer (Polyquaternium-7) |
| to 100.000 g | water |
| Component (B): | Hydrogen Peroxide Emulsion |
| 10.0 g | of cetylstearyl alcohol |
| 1.5 g | of cholesterol |
| 4.0 g | of sodium lauryl alcohol diethylene glycol ether |
| | sulfate, 28% aqueous solution |
| 35.0 g | of hydrogen peroxide, 35% aqueous solution |
| 0.3 g | of perfume |
| to 100.0 g | water |

Before use, 40 g of dye carrier composition (A) was mixed with 80 g of hydrogen peroxide emulsion (B), corresponding to an (A):(B) mixing ratio of 1:2, and 120 g of this mixture was applied to gray hair. After an exposure time of 20 minutes at room temperature, the hair was rinsed with water and then dried. The hair treated in this manner received a uniform light-blond coloration from hair roots to hair tips. The agent with a nacreous luster according to the invention was easy to apply to the hair and did not run off the hair.

EXAMPLE 3
Oxidation Hair Colorant, Creamy

| | |
|---|---|
| 5.50 g | of stearyl alcohol |
| 5.50 g | of behenyl alcohol |
| 10.00 g | of coco fatty acid monoethanolamide (Cocamide MEA) |
| 10.00 g | of ethylene glycol distearate |
| 1.00 g | of sodium myristyl sulfate (70% aqueous solution) |
| 1.20 g | of coco fatty acid isethionate |
| 8.00 g | of monoethanolamine |
| 2.30 g | of 1-hydroxyethyl-4,5-diaminopyrazole sulfate |
| 1.19 g | of amino-2-hydroxytoluene |
| 0.50 g | of cationic cellulose derivative (Polyquaternium-10) |
| 0.50 g | of keratin hydrolyzate |
| 0.50 g | of silk protein hydrolyzate |
| 0.50 g | of 2-amino-6-chloro-4-nitrophenol |
| 0.50 g | of disodium ethylenediaminetetraacetate |
| 0.30 g | of ascorbic acid |
| 0.10 g | of sodium sulfite |
| to 100.00 g | water |

Just before use, 50 g of the foregoing dye carrier composition was mixed with 50 g of a 12% aqueous hydrogen peroxide solution. The resulting mixture was then applied to natural medium-blond hair. After an exposure time of 30 minutes at 40° C., the hair was rinsed with water and dried. This gave a uniform, intense orange-red color shade.

EXAMPLE 4
Hair Colorant with Direct Dyes

| | |
|---|---|
| 0.500 g | of cetearyl alcohol |
| 3.000 g | of stearyl alcohol |
| 8.000 g | of behenyl alcohol |
| 8.500 g | of coco fatty acid monoethanolamide (Cocamide MEA) |
| 8.000 g | of ethylene glycol distearate |
| 2.000 g | of PEG-3 distearate |
| 2.000 g | of cocamidopropyl hydroxysultaine |
| 2.700 g | of sodium myristyl sulfate (70% aqueous solution) |
| 2.000 g | of isopropyl alcohol |
| 0.160 g | of HC Blue 12 |
| 0.170 g | of HC Yellow 13 |
| 0.012 g | of hydroxyethyl-2-nitrotoluidine |
| 0.035 g | of HC RED NO. 10 and HC RED NO. 11 (1:1) |
| 3.000 g | of dimethyldiallylammonium chloride/acrylamide |
| | copolymer (Polyquaternium-7) |
| 0.200 g | of cationic cellulose derivative (Polyquaternium-10) |
| to 100.000 g | water |

The creamy coloring composition with a nacreous luster was applied with gloves to washed and towel-dried natural blond hair and allowed to act for 20 to 25 minutes. Excess colorant was washed out with water and a shampoo. This gave a beautiful, lustrous, medium-blond shade.

EXAMPLE 5
Hair Colorant with Direct Dyes

| | |
|---|---|
| 7.0 g | of behenyl alcohol |
| 4.0 g | of stearyl alcohol |
| 9.0 g | of coco fatty acid diethanolamide (Cocamide DEA) |
| 10.0 g | of ethylene glycol distearate |
| 1.0 g | of sodium myristyl sulfate |
| 1.2 g | of coco fatty acid isethionate |
| 7.0 g | of ethanol, aqueous |
| 0.1 g | of hydroxyethyl-2-nitrotoluidine |
| 0.5 g | of HC RED NO. 10 and HC RED NO. 11 (1:1) |
| 0.2 g | of 2-amino-6-chloro-4-nitrophenol |
| 2.0 g | of dimethyldiallylammonium chloride/acrylamide copolymer |
| | (Polyquaternium-7) |
| 0.5 g | of cationic cellulose derivative (Polyquaternium-10) |
| to 100.0 g | water |

The creamy coloring composition with a nacreous luster was applied with gloves to washed and towel-dried natural blond hair and allowed to act for 20 to 25 minutes. Excess colorant was washed out with water and a shampoo. This gave a beautiful, lustrous, red-blond shade

Unless otherwise indicated, all percentages given in the present application are by weight.

1. Dye carrier composition containing oxidative and/or nonoxidative dyes, characterized in that it is free of fatty alcohol alkoxylates and that it contains a combination of
(a) at least one fatty alcohol with 14 to 60 carbon atoms,
(b) at least one alkanolamide,
(c) at least one fatty ester and
(d) at least one anionic surfactant,
the weight ratio of fatty alcohol (a) to alkanolamide (b) being equal to 1:4 to 4:1.

2. Dye carrier composition as defined in claim 1, characterized in that the fatty alcohol (a) and the alkanolamide (b) are present in a weight ratio from 1:1.7 to 2:1.

3. Dye carrier composition as defined in 1, characterized in that the weight ratio of fatty ester (c) to the anionic surfactant (d) is equal to 1:7to7:1.

4. Dye carrier composition as defined in claim 1, characterized in that the fatty alcohol (a) is selected from among cetyl alcohol, stearyl alcohol, behenyl alcohol, myristyl alcohol, isooctyl alcohol, isotridecyl alcohol, mixtures of C₃₀-C₅₀ alcohols and C₄₀-C₆₀ alcohols as well as a mixture of these compounds.

5. Dye carrier composition as defined in claim 1, characterized in that the alkanolamide (b) is selected from among the N-acyl derivatives of monoethanolamine or di-ethanolamine.

6. Dye carrier composition as defined in claim 1, characterized in that the fatty ester (c) is selected from among the monofatty acid or difatty acid esters of ethylene glycol, mono-fatty acid or difatty acid esters of polyethylene glycol and monofatty acid or difatty acid esters of glycerol.

7. Dye carrier composition as defined in claim 6, characterized in that the fatty acid ester (c) is selected from among ethylene glycol distearate, glyceryl dioleate, glyceryl stearate, glyceryl di-stearate, glyceryl dibehenate and PEG-3 distearate.

8. Dye carrier composition as defined in claim 1, characterized in that the ani-onic surfactant (d) is selected from among the salts and esters of carboxylic acids, alkyl ether sulfates, alkyl sulfates, fatty alcohol ether sulfates, sulfonic acids and the salts thereof, phosphate esters and the salts thereof and acylamino acids and the salts thereof.

9. Dye carrier composition as defined in claim 1, characterized in that the fatty alcohol (a) and the alkanolamide (b) are contained in said composition in a total amount from 6 to 20 weight percent each.

10. Dye carrier composition as defined in claim 1, characterized in that the fatty ester (c) and the anionic surfactant (d) are contained in said composition in a total amount rom 0.1 to 15 weight percent each.

11. Dye carrier composition as defined in claim 1, characterized in that it is free of monomeric quaternary ammonium compounds and cationic emulsifiers and surfactants.

12. Agent for oxidative coloring of hair, characterized in that it is obtained by mixing a dye carrier composition as defined in claim 1 with an oxidant.

13. Agent as defined in claim 12, characterized in that the oxidant is hydrogen peroxide.

14. Use of a combination of
(a) at least one fatty alcohol with 14 to 60 carbon atoms,
(b) at least one alkanolamide,
(c) at least one fatty ester and
(d) at least one anionic surfactant,
the weight ratio of fatty alcohol (a) to alkanolamide (b) being equal to 1:4 to 4:1, for creating a nacreous luster effect in dye carrier compositions and in colorants for keratin fibers, particularly human hair.

15. Use of a combination of
(a) 6 to 20 weight percent of at least one fatty alcohol with 14 to 60 carbon atoms,
(b) 6 to 20 weight percent of at least one alkanolamide,
(c) 0.1 to 15 weight percent of at least one fatty ester and
(d) 0.1 to 15 weight percent of at least one anionic surfactant,
the weight ratio of fatty alcohol (a) to alkanolamide (b) being equal to 1:4 to 4:1 for creating a nacreous luster effect in dye carrier compositions and in colorants for keratin fibers, particularly human hair.

16. Use as defined in claim 14, characterized in that the weight ratio of fatty alcohol (a) to alkanolamide (b) is equal to 1:1.7 to 2:1.
